(19) United States
(12) Patent Application Publication
(10) Pub. No.: US 2018/0207190 A1
(43) Pub. Date: Jul. 26, 2018

(54) COMPOSITION CONTAINING NAD FOR PREVENTING AND TREATING OBESITY OR IMPAIRED GLUCOSE TOLERANCE

(71) Applicant: UNIVERSITY OF ULSAN FOUNDATION FOR INDUSTRY COOPERATION, Ulsan (KR)

(72) Inventor: Min-Seon KIM, Seoul (KR)

(73) Assignee: UNIVERSITY OF ULSAN FOUNDATION FOR INDUSTRY COOPERATION, Ulsan (KR)

(21) Appl. No.: 15/745,552

(22) PCT Filed: Jul. 20, 2015
(86) PCT No.: PCT/KR2015/007491

§ 371 Date: Jan. 17, 2018

(30) Foreign Application Priority Data
Jul. 17, 2015 (KR) 10-2015-0101710

(51) Int. Cl. A61K 31/7084; A61K 9/00; A61P 3/04

(52) U.S. Cl. CPC A61K 31/7084; A61K 9/0019; A23L 33/13; A61P 3/04; A61K 9/0053

(57) ABSTRACT

A pharmaceutical composition containing nicotinamide adenine dinucleotide (NAD) as an active ingredient for preventing and treating obesity or impaired glucose tolerance, a food composition, and a method for preventing and treating obesity or impaired glucose tolerance using the same is disclosed. The NAD remedies an abnormal food intake pattern of an obese animal model induced by the intake of a high-fat diet and increases mobility, thereby exhibiting an effect of suppressing the weight increase due to the high-calorie intake and also showing an effect of improving glucose tolerance. In addition, it was verified that the NAD is capable of maintaining the above effects with even a much smaller quantity than an NAD precursor known in the prior art. Therefore, the composition containing NAD can be favorably used as a pharmaceutical composition or a food composition capable of effectively preventing and treating obesity or impaired glucose tolerance.

### FIELD OF THE DISCLOSURE

The present invention relates to a pharmaceutical composition containing nicotinamide adenine dinucleotide (NAD) as an active ingredient for preventing and treating obesity or impaired glucose tolerance, a food composition, and a method for preventing and treating obesity or impaired glucose tolerance using the same.

### DESCRIPTION OF RELATED ART

Nicotinamide adenine dinucleotide (NAD+) serves as an enzyme cofactor that mediates the transfer of hydrogen ions in an oxidative or reductive metabolic reaction (Berger, F., et al. (2004). “The new life of a centenarian: signaling functions of NAD+(P)” Trends in Biochemical Sciences 29(3): 111-118). NAD+ is converted into NAD+H through a glycolytic reaction catalyzed by glyceraldehyde 3-phosphate dehydrogenases and four stages of a tricarboxylic acid (TCA) cycle (Lin, S.-J. and L. Guarente (2003). “Nicotinamide adenine dinucleotide, a metabolic regulator of transcription, longevity and disease” Current Opinion in Cell Biology 15(2): 241-246). Also, NAD+ is converted into NAD+H during oxidation processes of fatty acids and amino acids in the mitochondria. To maintain a proper oxidation/reduction (redox) state, the NAD+H is reoxidized, and serves as an electron donor in oxidative phosphorylation and ATP synthesis processes in the mitochondria (Lin and Guarente, 2003).

In addition, NAD+ serves as an important cosubstrate in a biochemical reaction catalyzed by sirtuins and CD38 ectoenzymes. Sirtuins are class III-NAD+-dependent deacetylases, and play an important role in adaptive responses to nutritional and environmental stresses such as fasting, DNA damage, and oxidative stress. The sirtuins remove an acetyl group bound to lysine of a protein serving as a substrate and transfer the acetyl group to ADP-ribose. During a deacetylation process catalyzed by the sirtuins, NAD+ decomposes into nicotinamide.

A Preiss-Handler pathway for NAD+ synthesis, which starts from nicotinic acid (NA), is widely observed in unicellular organisms, and requires a subsequent amidation reaction of NA moieties by NAD+ synthetases (Preiss, J. and P. Handler (1958). “Biosynthesis of diphosphopyridine nucleotide” Journal of biological chemistry 233:493-500). The NAD+ biosynthesis in a mammal is carried out through the following four different pathways: NAD+ is synthesized through 1) de novo synthesis from tryptophan, 2) conversion from NA or nicotinamide, 3) conversion from nicotinamide riboside (NR), and 4) salvage from nicotinamide by a salvage pathway. NAD+ may also be newly synthesized from tryptophan in a mammal through a kynurenine pathway, but it is insufficient to maintain a normal NAD+ level. Most of NAD+ is synthesized from nicotinamide in humans (Rongvaux, A., et al. (2003). “Reconstructing eukaryotic NAD+ metabolism” Bioessays 25(7): 683-690), and the nicotinamide is released in a NAD+-dependent enzymatic reaction. A quantity of daily intake of nicotinamide nutritionally recommended is approximately 15 mg (Institute of Medicine Standing Committee on the Scientific Evaluation of Dietary Reference Intakes its Panel on Folate 1998), but NAD+ turnover is in a range of several grams in the liver alone (approximately 6.5 to 8.5 g of NAD+, which corresponds to approximately 1.5 g of nicotinamide) (Chiarugi, A., et al. (2012). “The NAD+ metabolome a key determinant of cancer cell biology” Nature Reviews Cancer 12(11):741-752). Therefore, the nicotinamide needs to be completely salvaged to maintain a NAD+ level in tissues, and for this purpose, an enzyme referred to as a nicotinamide phosphoribosyl transferase (Nampt) is required. The Nampt is a rate-limiting enzyme that catalyzes the transfer of a phosphoribosyl group from 5-phosphoribosyl-1-pyrophosphate (PRPP) to nicotinamide. As a result of the enzymatic reaction, nicotinamide mononucleotide (NMN) and pyrophosphate are generated (Revollo, J. R., et al. (2007). “The regulation of nicotinamide adenine dinucleotide biosynthesis by Nampt/PBEF/visfatin in mammals” Current Opinion in Gastroenterology 23(2): 164-170). In the next stage, the NMN is converted into NAD+ by a nicotinamide mononucleotide adenylyl transferase (Nmnat).

Meanwhile, the evidences suggesting that the aging has a significant influence on NAD+ biosynthesis in cell and organ levels, resulting in a decreased level of NAD+ in tissues of humans and rodents have been reported. In aged mice, an increase in PARP activities in the pancreas, white adipose tissue, liver, and skeletal muscles accompanies a decrease in NAD+ level, and this causes a decrease in SIRT1 activity and a decrease in function of the mitochondria. In aged rats, an increase in PARP activity caused by an increase in DNA damage in the heart, lungs, liver and kidneys causes a decrease in intracellular NAD+ level, and this leads to decreased SIRT1 activity and decreased mitochondrial activity. Also, a decrease in NAD+ level and a decrease in SIRT1 activity caused by the increased PARP activity in the aged rats' brains are observed (Braidy, Guillemin et al. 2011). Similar to these results, Liu et al. also reported that the Nampt activity and NAD+ level decrease in the aged animals' brains (Liu, L.-Y., et al. (2012). “Nicotinamide phosphoribosyltransferase may be involved in age-related brain diseases” PloS one 7(10): e44933).

Therefore, according to the results of research thus reported, because an appropriate cellular NAD+ content is very important for normal metabolic functions in tissues, various therapeutic strategies have been proposed to increase an NAD+ level in cells. First, a plan for increasing an NAD+ level by supplementing NR as an NAD+ precursor was proposed. However, the NAD+ level increases in peripheral tissues such as liver and skeletal muscles, but does not increase in the brain (CantoHoutkooper et al. 2012). Another research group used NMN which is a Nampt reaction product and an important NAD+ precursor. The intraperitoneal (IP) administration of NMN to diabetic mice fed a high-fat diet (HFD-fed) successfully increases an NAD+ level in the liver, adipose tissues, and skeletal muscles (Yoshino, Mills et al. 2011). Also, the administration of NMN to the diabetic mice restores damaged glucose tolerance. These results show that the NMN administration improves NAD+ deficiency in the diabetes induced by the high-fat diet (HFD) (Yoshino, Mills et al. 2011). However, an effect of the NMN administration on an increase in NAD+ level in the hypothalamus remains to be studied, and a transport mechanism of NMN into cells is not identified yet.

Second, because PARP-1 is an important NAD+ consumer in cells, pharmaceutical inhibition of PARP may increase an in vitro and in vivo NAD+ level in the cells and enhance SIRT1 activity. Therefore, development of PARP inhibitors is considered to be one therapy for diseases accompanied by metabolic dysregulation.

Third, there is a method of inducing an increase in NAD(P)+/NAD(P)H ratio by converting NAD(P)H into NAD(P)+ through a redox reaction mediated by NAD(P)H:quinone oxidoreductase 1 (NQO1). To prevent and treat diseases such as obesity, diabetes, metabolic syndrome, and degenerative disease which are associated with a decrease in the NAD(P)+/NAD(P)H ratio and caused due to excessive intake or change of energy in a redox pathway, NQO1 may be developed as a plan for increasing the NAD(P)/NAD(P)H ratio in these disease models (Mazence, 2007 Aug. 21, Method for controlling NAD(P)/NAD(P)H ratio by oxidoreductase., 10-2007-0082557). It was reported that a pyrano-1,2-naphthoquinone compound (βL), which is one of NQO1 activating factors, increase the NAD+, NAD+/NADH, and NADP+/NADPH ratios. These metabolic effects of the compounds to increase the NQO1 activity remain to be studied.

It was known from an in vivo experiment that the apoptosis induced by oxidative stress is reduced when cultured neuronal cells, astrocytes, and cardiac myocytes are treated with NAD+, and it was also reported from an in vivo experiment using rodents that the exogenous NAD+ administration improves ischemic brain damage and cardiomegaly (Pillai, V. B., et al. (2010). “Exogenous NAD+ blocks cardiac hypertrophic response via activation of the SIRT3-LKB1-AMP-activated kinase pathway” Journal of Biological Chemistry 285(5): 3133-3144; Zheng, C., et al. (2012). “NAD+ administration decreases ischemic brain damage partially by blocking autophagy in a mouse model of brain ischemia” Neuroscience Letters 512(2):67-71). However, there is still no report on research results related to a method of administering NAD+ itself in an animal level so as to treat obesity and type II diabetes.

Accordingly, the present inventors have found that direct systemic administration of NAD+ is effective in effectively improving symptoms of obesity and glucose tolerance. Therefore, the present invention has been completed based on these facts.

### SUMMARY OF THE INVENTION

Therefore, it is an aspect of the present disclosure to provide a pharmaceutical composition for preventing and treating obesity or impaired glucose tolerance using nicotinamide adenine dinucleotide (NAD).

It is another aspect of the present disclosure to provide a food composition for preventing and improving obesity or impaired glucose tolerance using NAD.

It is still another aspect of the present disclosure to provide a method for preventing and treating obesity or impaired glucose tolerance using NAD.

To solve the above problems, according to an aspect of the present invention, there is provided a pharmaceutical composition containing nicotinamide adenine dinucleotide (NAD) or a pharmaceutically acceptable salt thereof as an active ingredient for preventing and treating obesity or impaired glucose tolerance.

According to one exemplary embodiment of the present invention, the NAD may control a food intake pattern of an obese patient, which includes a food intake time and cycle, to reduce the intake of food.

According to one exemplary embodiment of the present invention, the NAD may increase physical activity of the obese patient.

According to one exemplary embodiment of the present invention, the pharmaceutical composition may be administered in a form of intraperitoneal, intravascular or oral administration.

According to one exemplary embodiment of the present invention, the NAD may be administered at a dose of 0.1 to 100 mg per unit weight (kg) of a target subject when the pharmaceutical composition is intraperitoneally administered.

According to one exemplary embodiment of the present invention, the NAD may be administered at a dose of 0.1 to 100 mg per unit weight (kg) of a target subject when the pharmaceutical composition is intravascularly administered.

According to one exemplary embodiment of the present invention, the NAD may be administered at a daily dose of 1 to 1,000 mg per unit weight (kg) of a target subject when the pharmaceutical composition is orally administered.

According to another aspect of the present invention, there is provided a food composition containing NAD or a sitologically acceptable salt thereof for preventing and improving obesity or impaired glucose tolerance.

According to one exemplary embodiment of the present invention, the NAD may serve to control a food intake pattern of an obese patient, which includes a food intake time and cycle, to reduce the intake of food.

According to still another aspect of the present invention, there is provided a method for preventing and treating obesity or impaired glucose tolerance, which includes administering NAD to a non-human mammal subject.

According to one exemplary embodiment of the present invention, the administration may be performed by intraperitoneal, intravascular or oral administration.

According to one exemplary embodiment of the present invention, the NAD may be administered at a dose of 0.1 to 100 mg per unit weight (kg) of a target subject when the pharmaceutical composition is intraperitoneally administered.

According to one exemplary embodiment of the present invention, the NAD may be administered at a dose of 0.1 to 100 mg per unit weight (kg) of a target subject when the pharmaceutical composition is intravascularly administered.

According to one exemplary embodiment of the present invention, the NAD may be administered at a dose of 1 to 1,000 mg per unit weight (kg) of a target subject when the pharmaceutical composition is orally administered.

According to one exemplary embodiment of the present invention, the administration may be performed once to three times a day.

The NAD+ administration according to the present invention improves an abnormal food intake pattern in an obese animal model induced by the intake of a high-fat diet and increases mobility, thereby exhibiting an effect of suppressing the weight gain caused due to the high-calorie intake and showing an effect of improving symptoms of glucose tolerance as well. Also, it is verified that the NAD of the present invention can maintain the aforementioned effects even when used at a quantity much smaller than that of NMN, which is an NAD+ precursor known in the prior art. Therefore, the composition containing the NAD of the present invention can be favorably used as a pharmaceutical composition or a food composition capable of effectively preventing and treating obesity or impaired glucose tolerance.

### BRIEF DESCRIPTION OF THE DRAWINGS

FIG. 1 shows that NAD+ levels in the plasma and hypothalamus decrease in mice fed a high-fat diet. To measure an NAD+ level, C57BL/6 mice fed a high-fat diet (HFD) or a normal diet (ND) for 20 weeks are fasted for 5 hours, and sacrificed to collect plasma and hypothalamus, and an NAD+ level is measured using high performance liquid chromatography (HPLC). It was confirmed that the NAD+ levels in both the plasma and hypothalamus of the mice fed the HFD for 20 weeks were significantly reduced, compared to those of the mice fed the ND (P<0.05).

FIG. 2 shows experimental results showing effects of single intravascular administration of NAD+ and NMN on food intake and body weight. (A) After 0.2, 1, and 2 pmol of NAD+ was intravascularly administered once to C57BL/6 mice that were fasted overnight, the C57BL/6 mice were freely fed so that changes in feed intake and body weight were observed for 24 hours. The food intake was significantly reduced in the mice to which the NAD+ was administered, compared to the mice to which saline was administered. A significant effect on a decrease in the food intake was observed from 2 hours after the NAD+ administration and maintained even after 24 hours. (B) shows results of investigating a change in body weight for 24 hours after the NAD+ administration. The weight gain was significantly suppressed for 24 hours in the mice to which NAD+ was injected at a dose of 0.2 pmol, compared to the mice to which saline was injected. (C) To compare an effect of NMN versus NAD+, 10, 100, and 1,000 pmol of NMN was intraventricularly injected once to C57BL/6 mice that were fasted overnight. The food intake was reduced in the mice to which 10 pmol of NMN was injected, compared to the mice to which saline was injected, but an effect of NMN on suppression of the food intake was insignificant, compared to that of NAD+(0.2 pmol). (D) There was no significant difference in the change in the body weight for 24 hours after the injection between a control group and an NMN-treated group. The aforementioned research results showed that the intraventricular administration of NAD+ induced a more effective and sustained decrease in feed intake and body weight compared to NMN administration, even at a small dose that was 1/20-fold of that of NMN.

FIG. 3 shows experimental results showing an effect of single intraperitoneal (IP) injection of NAD+ and NMN on food intake. (A) When the feed intake and body weight were measured for 24 hours after NAD+(dose: 0.3, 1, and 3 mg/kg) was intraperitoneally administered once to C57BL/6 mice that were fasted overnight, at 4 hours after the NAD+ intraperitoneal administration, the food intake was significantly reduced in the mice to which NAD+ was injected, compared to the mice to which saline was injected. (B) At 24 hours after the NAD+ administration, the mice to which 1 mg/kg of NAD+ was injected had a significantly reduced food intake, compared to the mice to which saline was injected. (C) 30, 100, and 300 mg/kg of NMN was intraperitoneally injected once to C57BL/6 mice that were fasted overnight. The mice to which 300 mg/kg of NMN was injected had a significantly reduced food intake at 4 hours after the administration, compared to the control to which saline was injected. (D) The NMN-injected mice did not have a reduced food intake for 24 hours after the administration, compared to the control. The aforementioned research results showed that the intraventricular administration of NAD+ induced a more effective decrease in feed intake and body weight compared to NMN administration, even at a small dose that was about 1/300-fold of that of NMN.

FIG. 4 shows experimental results showing an effect of chronic NAD+IP injection on body weight. NAD+(0.3 mg/kg/day) was intraperitoneally administered once a day for 4 weeks to four groups of mice (that is, a group of ND-fed mice to which saline was IP injected, a group of ND-fed mice to which NAD+ was IP injected, a group of HFD-fed obese mice to which saline was IP injected, and a group of HFD-fed obese mice to which NAD+ was IP injected) immediately before lights went off. A significant difference in body weights between an NAD+-injected group and a saline-injected group was not observed in the ND-fed normal mice. On the other hand, the body weights of the HFD-fed obese mice were significantly reduced in the NAD+-injected group, compared to the saline-injected group. Such research results showed that the NAD+ treatment did not induce a weight loss of the mice with a normal body weight, but induced a weight loss of the mice only in an obese condition.

FIG. 5 shows experimental results showing an effect of chronic IP injection of NAD+ on a circadian rhythm of feed intake. For this purpose, the three experimental groups (that is, a group of ND-fed mice to which saline was IP injected, a group of HFD-fed obese mice to which saline was IP injected, and a group of HFD-fed obese mice to which NAD+ was IP injected) shown in FIG. 4 were put into continuous lab animal monitoring system (CLAMS) cages (Oxymax), and feed intake patterns of the mice were analyzed for 24 hours. (A) The ND-fed mice to which saline was injected exhibited an evident circadian rhythm of feed intake in which the mice were usually fed during the nighttime (corresponding to the daytime in humans) and hardly fed during the daytime. On the other hand, it was shown that the circadian rhythm of feed intake was disturbed as the feed intake during the daytime as well as the nighttime increased in the HFD-fed obese mice. This is similar to an increased nighttime food intake as observed in obese humans. The NAD+ injection to the obese mice did not significantly reduce the nighttime feed intake, but significantly reduced the daytime feed intake. Such research results showed that the NAD+ treatment was effective in reducing the disturbance of circadian rhythm of feed intake in individuals with obesity. (B) When a feed intake frequency was analyzed, the HFD-fed obese mice had an increased food intake frequency and particularly a remarkably increased food intake frequency during the daytime compared to the ND-fed normal mice. Also, the NAD+ treatment suppressed a high food intake frequency of the obese mice to a normal mouse level. Therefore, it was proven that the NAD+ treatment was effective in reducing an increased food intake frequency in individuals with obesity.

FIG. 6 shows experimental results showing an effect of habitual IP injection of NAD+(0.3 mg/kg/day) on physical activity. (A) Three groups (that is, an IP saline-injected group of mice fed the ND, an IP saline-injected group of mice fed the HFD, and an IP NAD+-injected group of mice fed the HFD) were put into CLAMS cages so that the physical activity of the mice was determined for 24 hours. An evident circadian rhythm of physical activity was observed: the physical activity of the ND-fed normal control to which saline was injected was significantly higher during the nighttime (corresponding to the daytime in humans) than during the daytime. On the other hand, it was shown that the HFD-fed group of obese mice to which saline was administered had a disrupted circadian rhythm of physical activity, exhibiting significantly reduced nighttime physical activities compared to the control. The 4-week IP injection of NAD+ to the obese mice remarkably restored the nighttime mobility of the mice which had been reduced. (B) is a graph of quantified physical activity of mice. Such research results proved that the NAD+ administration effectively reduced the disturbance of circadian rhythm of physical activity in individuals with obesity.

FIG. 7 shows experimental results showing an effect of NAD+ administration on glucose tolerance. For this purpose, NAD+(0.3, 1, 3 mg/kg) was intraperitoneally administered once to normal C57BL/6 mice that were fasted overnight, and glucose (2 g/kg) was orally administered thereto after 30 minutes. Blood was collected from tail veins of the mice immediately before the glucose administration and 15, 30, 60, and 120 minutes after the glucose administration, and blood glucose levels were measured using a glucometer. The blood glucose levels of the mice to which NAD+ was injected were significantly reduced at 15 and 30 minutes after the glucose administration, compared to the mice to which saline was injected. Such research results showed that the NAD IP administration may improve glucose tolerance.

### DETAILED DESCRIPTION OF THE INVENTION

Obesity is associated with aging and an increase in energy intake, and a calorie restriction improves health and lifespan in various organisms (Colman, R. J., et al. (2009). “Caloric restriction delays disease onset and mortality in rhesus monkeys” Science 325(5937): 201-204). In recent years, NAD+ has attracted attention as a main regulator of metabolism, stress resistance and lifespan. A decrease in NAD+ in the hypothalamus caused due to the excessive energy intake and aging is considered to contribute to the onset of metabolic disorders accompanied by the obesity and aging. However, because there is still no research on a therapeutic effect of administering NAD+ itself on food intake, body weight, and glycometabolism, the present inventors have tested an effect of directly administering NAD+.

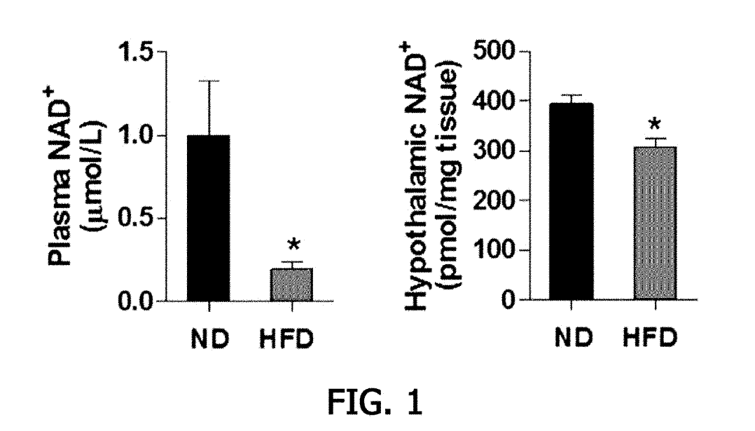

First, the present inventors have measured NAD+ levels in the plasma and hypothalamus of mice fed a high-fat diet (HFD), and compared the NAD+ levels with those of mice fed a normal diet (ND). As a result, it was observed that the NAD+ levels in the blood and the hypothalamus of the brain, which is the control center for appetite and body weight, were significantly reduced in the obese mice which were fed the HFD for 20 weeks, compared to the mice fed the ND (FIG. 1). Therefore, it was proven that the NAD+ deficiency is accompanied by the obesity caused by a high-fat diet.

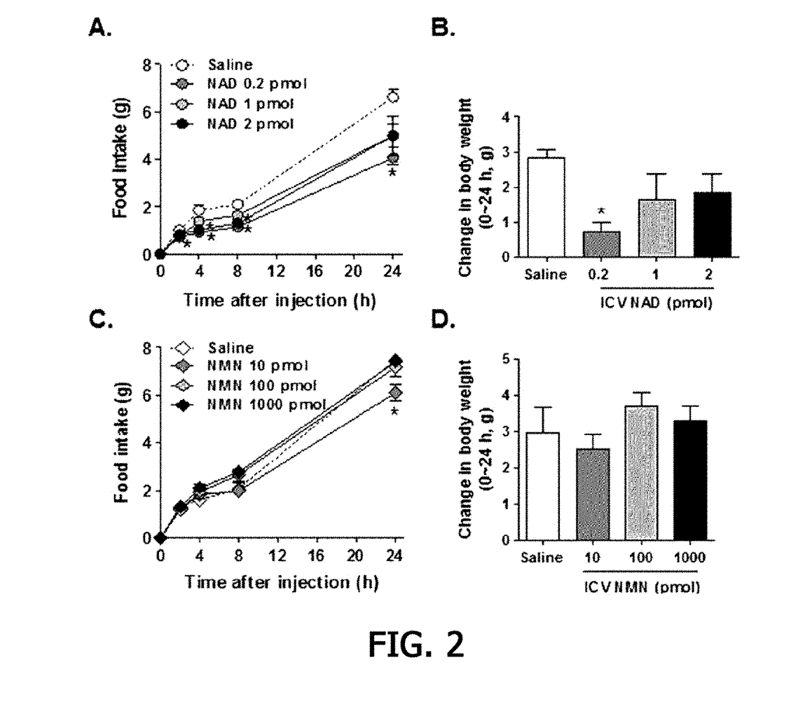

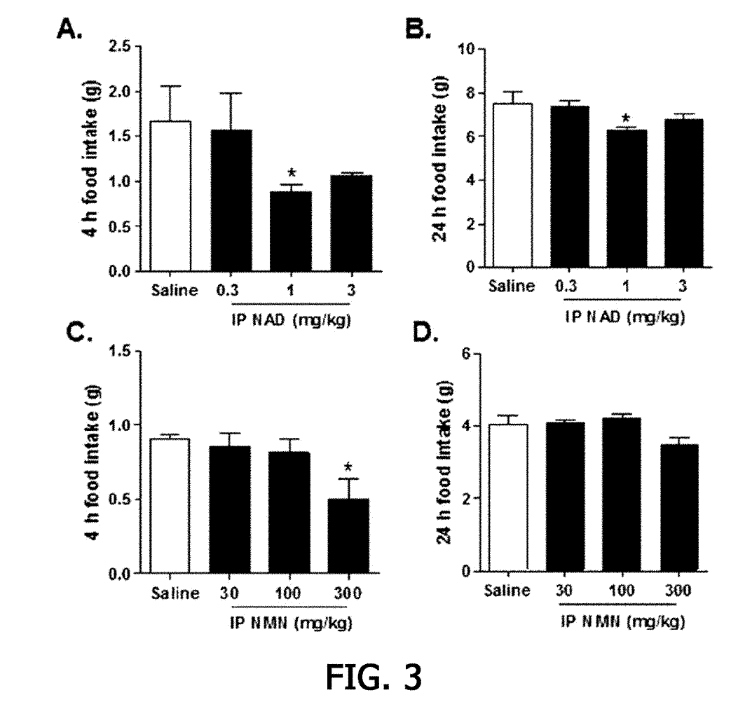

The present inventors have investigated an effect of single intravascular or intraperitoneal administration of NAD+ on food intake and body weight. The intravascular injection of NAD+ significantly reduced the food intake and weight gain for 24 hours after the injection, compared to the saline injection (FIG. 2). The systemic NAD+ administration through the intraperitoneal injection was as effective in reducing the food intake as the intravascular injection (FIG. 3). Interestingly, a very small amount (at most 1/100-fold of an effective dose of NMN as a precursor) of NAD+ could significantly suppress the food intake and body weight. Also, the effect of NAD+ was maintained for 24 hours after the intraperitoneal injection, but the effect of NMN was not maintained. That is, the intravascular administration of NAD+(0.2 pmol) reduced the body weight for 24 hours after the injection, but the ICV administration of NMN did not cause a change in body weight. Particularly, a smaller dose of NMN (10 pmol) and NAD+(0.2 pmol) was more effective in terms of an appetite suppression action than a higher dose of NMN (100 and 1,000 pmol) and NAD+(1 and 2 pmol). Because most toxic effects are proportional to the dose, the aforementioned results show that the suppression of food intake induced by NAD+ and NMN is not due to the non-specific toxicity. Therefore, it is important to determine the most effective dose of the NAD+ and NMN for therapeutic purposes.

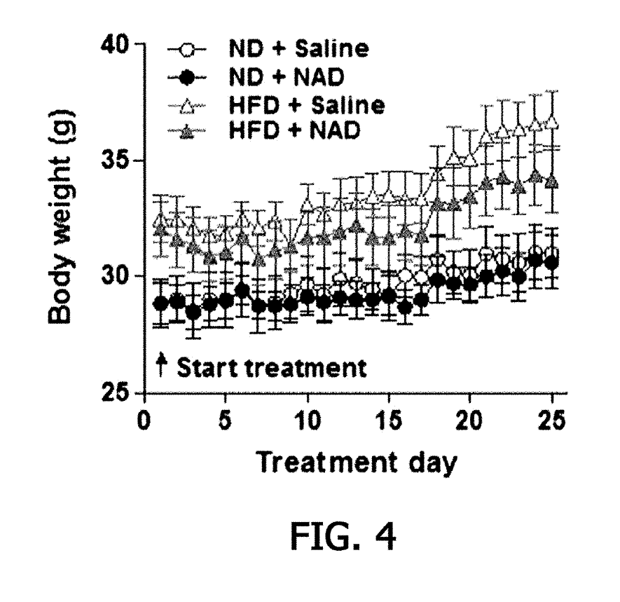

Next, the present inventors have examined an effect of chronic administration (NAD+0.3 mg/kg, intraperitoneally injected once a day for 4 weeks) of NAD+ on body weight by employing mice fed a normal diet (chow-diet) and mice fed a high-fat diet as subjects. As a result, a significant effect of NAD+ on weight loss was observed in mice whose obesity had been induced by the high-fat diet but not in the normal mice fed the normal diet (FIG. 4). This indicates that the NAD intake can induce weight lose especially in obese subjects.

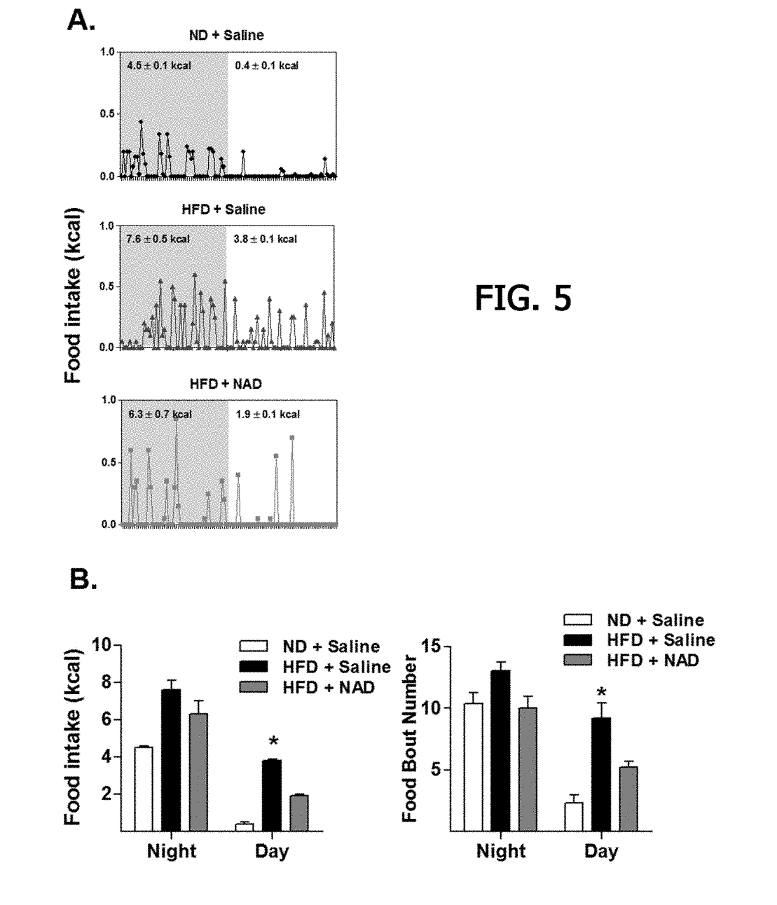

The loss of circadian rhythm of food intake, that is, an increase in daytime food intake (corresponding to a late-night meal in humans) and an increase in daytime food intake frequency, was observed in the diet-induced obese mice. In particular, the treatment of the obese mice with NAD+ significantly reduced the quantity and frequency of daytime food intake (FIG. 5), and this indicates that the NAD+ treatment may be able to restore the disrupted circadian rhythm of food intake for obese patients.

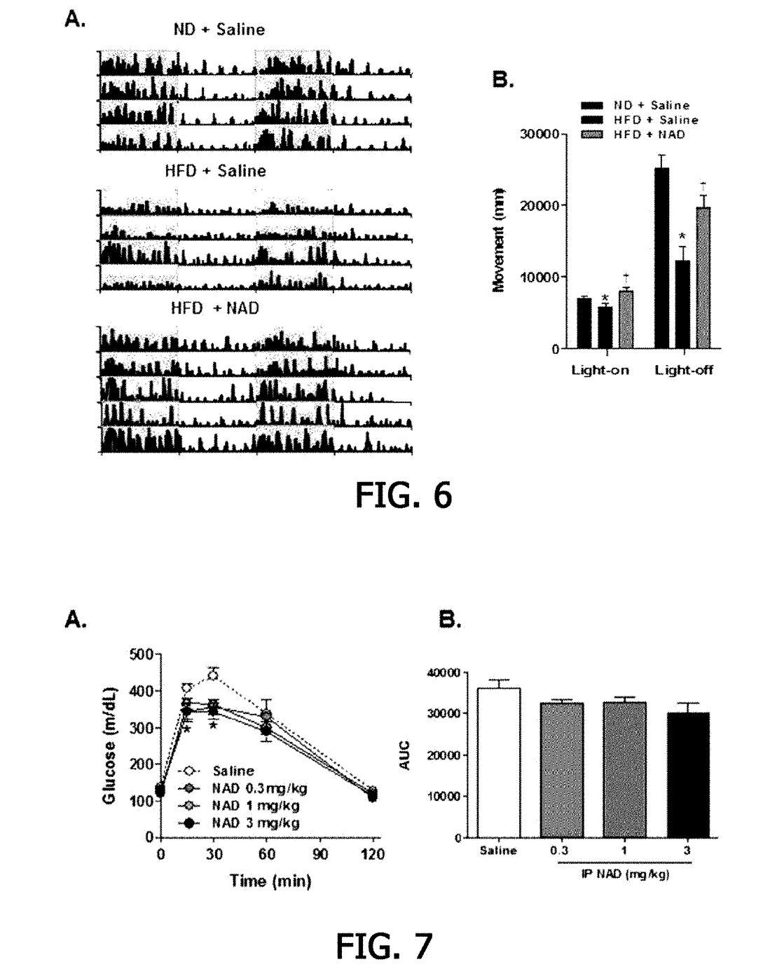

In addition, the 4-week administration of NAD+ also restored the physical activity of the diet-induced obese mice, which had been reduced during the nighttime (FIG. 6). Therefore, the increased physical activity may be another mechanism for an anti-obesity effect of the NAD+ intake. Also, the chronic injection of NAD+ did not cause any notable side effects, and this indicates that the habitual systemic NAD+ treatment is safe. From these results, it can be seen that the systemic NAD+ administration causes a decrease in food intake and an increase in mobility, thereby preventing the weight gain caused by high-calorie intake.

Also, the present inventors have examined an effect of single administration of NAD+ on a blood glucose level during a glucose challenge test. When NAD+ was intraperitoneally administered to mice before glucose administration, the blood glucose level was significantly reduced at 15 minutes and 30 minutes after the glucose administration. These research results suggest that the NAD+ administration may improve glucose tolerance. Therefore, the present invention may provide a pharmaceutical composition containing nicotinamide adenine dinucleotide (NAD) or a pharmaceutically acceptable salt thereof as an active ingredient for preventing and treating obesity or impaired glucose tolerance. The term “prevention” used in this specification means that a therapeutic agent (for example, a prophylactic or therapeutic agent) or a combination of therapeutic agents prevents manifestation of symptoms of obesity or impaired glucose tolerance in a target subject or prevent recurrence or development of obesity or impaired glucose tolerance when administered to the target subject. The term “treatment” used in this specification means to improve or regulate symptoms or one or more physical parameters of patients with obesity or impaired glucose tolerance, or delays the onset or progression of the obesity or impaired glucose tolerance, regardless of whether the patients recognize these changes. The term “pharmaceutically acceptable” used in this specification generally refers to a composition that is physiologically acceptable and does not cause gastrointestinal disorders, and allergic reactions such as dizziness, or similar reactions when administered to animals. A pharmaceutical composition of the present invention may include one or more pharmaceutically acceptable carriers, excipients or diluents. Examples of the carriers, excipients and diluents may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oils. Also, the pharmaceutical composition of the present invention may further include a filler, an anticoagulant, a lubricant, a wetting agent, a flavoring agent, an emulsifying agent, a preservative, or the like. Carriers suitable for use may include saline, phosphate-buffered saline, an aqueous medium including a minimum essential medium (MEM) or MEM of a HEPES buffer solution, but the present invention is not limited thereto.

Also, the pharmaceutical composition of the present invention may be formulated using methods known in the related art to provide a fast, sustained or delayed release of an active ingredient after the pharmaceutical composition is administered to a mammal. A formulation may be in the form of a powder, a granule, a tablet, an emulsion, syrup, an aerosol, a soft or hard gelatin capsule, a sterile injectable solution, a sterile powder, or the like. The pharmaceutical composition of the present invention may be administered through a route of intramuscular, subcutaneous, percutaneous, intravenous, intranasal, intraperitoneal or oral administration, preferably administered through a route of intramuscular or subcutaneous administration. The dose of the composition may be properly chosen according to various factors such as a route of administration, the age, sex, and body weight of an animal, and the severity of a disease.

The pharmaceutical composition of the present invention may be formulated into a variety of the following forms of oral or parenteral administration, but the present invention is not limited thereto. First, solid preparations for oral administration include tablets, pills, powders, granules, hard or soft capsules, and the like. Such a solid preparation may be compounded by mixing at least one excipient with the active ingredient of the present invention. Also, lubricants such as magnesium stearate, talc, and the like may be used in addition to the simple excipients. Liquid preparations for oral administration include suspensions, liquids for internal use, emulsions, syrup, and the like and may contain various excipients in addition to a generally used simple diluent such as water, liquid paraffin, or the like. Also, the pharmaceutical composition of the present invention may be parenterally administered. In this case, the parenteral administration is performed using a method of injecting a subcutaneous injection, an intravenous injection, an intramuscular injection, an intrathoracic injection, or the like. In this case, to prepare the composition into a formulation for parenteral administration, the active ingredient of the present invention may be mixed with a stabilizing agent or a buffer in water to prepare a solution or a suspension, and the solution or suspension may be prepared into unit dosage forms provided in ampoules or vials. Preparations for parenteral administration include sterile aqueous solutions, non-aqueous solvents, suspensions, emulsions, freeze-dried preparations, suppositories, and the like. Propylene glycol, polyethylene glycol, a vegetable oil such as olive oil, or an injectable ester such as ethyl oleate may be used as the non-aqueous solvent or the suspension.

Also, the pharmaceutical composition of the present invention may be administered to a mammal such as a mouse, a rat, a domestic animal, a human, and the like through various routes of administration. For example, the pharmaceutical composition may be administered orally, or rectally, or by intravenous, intramuscular, subcutaneous, cervical epidural or intra-cerbroventricular injection. The NAD of the present invention may be administered using methods properly selected according to the age, sex, and body weight of a patient.

According to another aspect, the NAD of the present invention may be used in a functional food composition. The food composition according to the present invention may be expected to have an anti-obesity or glucose tolerance-improving effect of NAD. The functional food composition of the present invention may be prepared by further blending another physiologically active substance, that is, a natural antioxidant substance or the like whose safety is verified, to multiply the effects. For example, the food composition of the present invention may be prepared into any one formulation selected from the group consisting of a tablet, a granule, a powder, a capsule, a liquid solution, and a pill, but the present invention is not limited thereto. The shape of the food composition of the present invention is not particularly limited. For example, the food composition may be prepared into forms such as a liquid food, an enteral nutritious food, a health food, and a food for infants or children in addition to the typical forms. For constant intake, the food composition may be prepared into forms such as cooked rice, various condiments, mixed oils or processed fat products such as margarine, shortening, mayonnaise, dressing, and the like. Also, the food composition may be prepared into any form typically used in the related art, such as a solid form, a semi-solid form, a gel form, a liquid form, a powdery form, or the like. Also, the food composition of the present invention may be prepared into cookies, processed foods, mixed oils, dairy products, drinks, vitamin complexes, health functional foods, or the like for commercialization. Further, the food composition of the present invention may contain various nutrients, vitamins, electrolytes, flavoring, coloring and enhancing agents, pectic acid, alginic acid, organic acids, protective colloid thickening agents, pH regulators, stabilizing agents, preservatives, glycerin, alcohol, carbonating agents used in carbonated drinks, and the like in addition to glucoproteins of the present invention. Here, such components may be used alone or in combination.

In addition, the present invention is directed to provide a novel method for treating obesity or impaired glucose tolerance, confirms that the method is effective in excellently treating obesity or impaired glucose tolerance by directly administering NAD itself, compared to conventional methods, and verifies the optimum dose of NAD according to a route of administration.

The optimum dose of NAD according to the route of administration may be administered through intraperitoneal, intravascular or oral administration, as described above. For the intraperitoneal and intravascular administration, NAD is preferably administered at a dose of 0.1 to 100 mg per unit weight (kg) of a target subject. For the oral administration, NAD is preferably administered at a dose of 0.1 to 1,000 mg per unit weight (kg) of the target subject.

According to one exemplary embodiment of the present invention, a therapeutic effect of NAD on obesity or impaired glucose tolerance was verified particularly by administering NAD to mice subjects. It was confirmed that the dose of NAD that may give such an effect is in a range of 0.03 to 1,000 mg/kg. In this way, a treatment concentration of a pharmacological substance identified through an animal experiment may be used to estimate a treatment concentration of the pharmacological substance applicable to humans through the following equation know in the related art.

Human-applied concentration (mg/kg)=animal-applied concentration (mg/kg)×animal-applied Km index

Here, the Km index is a predetermined value for conversion into a body surface area for a subject. In this case, the Km indexes of a human adult, a human child, a mouse and a rat are set to 37, 25, 3, and 6, respectively. Therefore, when the concentration of NAD applied to a mouse subject according to the present invention is converted into a concentration of NAD to be applied to the human (adult) by using such an equation, it can be seen that a human (adult) may be treated with NAD at a dose of 0.1 mg/kg to 3,000 mg/kg. When NAD is intraperitoneally or intravascularly administered to the human (adult), the human (adult) is preferably treated with NAD at a dose of 0.1 mg/kg to 300 mg/kg. For oral administration, the human (adult) is preferably treated with NAD at a dose of 0.1 mg/kg to 3,000 mg/kg. More preferably, when NAD is intraperitoneally or intravascularly administered to the human (adult), the human (adult) may be treated with NAD at a dose of 0.1 mg/kg to 100 mg/kg, and for oral administration, the human (adult) may be treated with NAD at a dose of 0.1 mg/kg to 1,000 mg/kg.

When the dose of NAD according to the respective routes of administration is greater than the above-described ranges, a therapeutic effect may be insufficient and other side effects may be caused in the body. Therefore, it is desirable that NAD is administered within these ranges.

### EXAMPLES

Hereinafter, the present invention will be described in further detail with reference to examples thereof. However, it will be apparent to those skilled in the art that the following examples are merely given herein to describe the present invention more fully, but are not intended to limit the scope of the present invention.

### Example 1

Experimental Materials and Methods

<1-1> Laboratory Animals

Mature male C57BL/6 mice were purchased from ORIENT BIO Inc. (Gyeonggi-do, Republic of Korea). Unless stated otherwise, the mice were freely fed a standard diet (Cargill Agri Purina, Inc., Seoul, Republic of Korea). To establish a diet-induced obesity (DIO) model, the mice were fed a HFD (60% fats, Research Diet Co., New Brunswick, N.J.) for 20 weeks. The animals were raised at a controlled temperature (22±1° C.) under a 12-hour light/dark cycle (a light condition from 08:00 a.m. to 8:00 p.m.) For experiments of single administration of NAD+ and NMN, 8-week-old mice were fasted overnight, and NAD+(purchased from Sigma, administered at 0.3, 1 and 3 mg/kg) or NMN (purchased from Sigma, administered at 30, 100, and 300 mg/kg) was intraperitoneally administered to the mice between 09:00 a.m. and 10:00 a.m. For [symbol] chronic NAD+ treatment, NAD+(0.3 mg/kg body weight/day) was intraperitoneally injected once to the mice immediately before the lights went off for 4 weeks.

<1-2> Intravascular Cannulation and NAD+ Administration

Permanent 26-gauge stainless steel cannulae were surgically inserted into the third ventricles of mice (a cannula insertion position: 1.8 mm posterior to bregma and 5.0 mm below the sagittal sinus). For surgery, the mice were anesthetized with a mixture of Zoletil and Rumpun (2:1 v/v, 10 μL/g body weight). The exact cannulation positions of the cannulae were confirmed through a positive dipsogenic response after angiotensin II (50 ng) was administered. Only the mice in which the cannulae were precisely positioned were used for data analyses. After a recovery period of 7 days after the surgery, the mice were daily touched for a predetermined time over a week to minimize a stress response to the experiments. Immediately before administration, NAD+ and NMN were dissolved in 0.9% saline and used. The NAD+ and NMN were dissolved in 2 μL of saline for intravascular administration.

<1-3> Feeding Study

A predetermined dose of NAD+ or NMN was administered to C57BL/6 mice, which were fasted overnight, during an early light phase (09:00 to 11:00) by using a given method. The same amount of a vehicle (i.e., physiological saline) was administered to the control mice. After the injection, the food intake and body weight of the mice were observed for 24 hours.

<1-4> Evaluation of Circadian Rhythm of Feed Intake and Physical Activity

The feed intake and physical activity of mice were measured using a comprehensive laboratory animal monitoring system (CLAMS, Columbus Instruments) (n=4 to 5). The light and feeding conditions were maintained under the same conditions as in a home cage. Water was freely provided during an observation period.

<1-5> Oral Glucose Tolerance Test

For an oral glucose tolerance test, the mice were fasted overnight, 2 g/kg (body weight) of glucose was orally administered (by oral gavage), and blood glucose levels were measured immediately before oral administration (0 minute) and 15, 30, 60, and 120 minutes after the oral administration. 30 minutes prior to glucose administration, NAD+(0.3, 1 and 3 mg/kg) was intraperitoneally administered.

<1-6> NAD+ Measurement

NAD+ was extracted from 100 μL of plasma and hypothalamus tissue using 100 μL of 1 M HClO₄, and neutralized by adding 66 μL of 3 M K₂CO₂. After centrifugation (4° C., 13,000 g) for 15 minutes, 20 mL of a supernatant was loaded onto a HPLC column (AHima HPC 18AQ 5 mM, 15×4.6 cm).

When HPLC was run at a rate of 1 mL/min, NAD+ produced a sharp peak at 10-minute time point. The NAD+ level was quantified based on the peak area in comparison with a standard curve, and corrected with the wet weight of the tissue. This refers to the method provided in the previous paper by Imai Shin (Ramsey, Mills et al. 2008, Yoshino,

Mills et al. 2011).

<1-7> Statistical Analysis Data are represented as means±standard error of the mean (SEM). The statistical analysis was performed using IBM SPSS (Chicago, Ill.). A statistical significance between groups was verified using a one-way analysis of variance (ANOVA) test, a post hoc LSD test, or an unpaired Student's t-test. Statistical significance was defined as P<0.05.

### Example 2

Effect of NAD+ Administration on Food Intake and Body Weight in Obese Animal Model

To measure an NAD+ level in an animal whose obesity was induced by dietary intake, the plasma and hypothalamus were taken from C57BL/6 mice fed a high-fat diet (HFD) or a normal diet (ND) on week 20 after the feeding so as to perform HPLC to measure an NAD+ level (FIG. 1). A significant decrease in NAD+ levels in both the plasma and hypothalamus was observed in the mice fed the HFD for 20 weeks, compared to the mice fed the ND (P<0.05).

To examine an effect of the NAD+ administration on the food intake in an obese animal model, NAD+ was ICV or IP administered, and NMN which is a NAD+ precursor known in the prior art was used as a target for a comparative experiment. First, to check an effect of the NAD+ administration through ICV injection on the food intake in the obese animal model, 0.2, 1, and 2 pmol of NAD+ was intravascularly administered once to the ND-fed C57BL/6 mice that were fasted overnight. The mice to which NAD+ was injected had a reduced food intake, compared to the control which is the mice to which saline was injected (FIG. 2A), and a decrease in food intake was started at 2 hours after the NAD+ administration, and maintained even at 24 hours after the NAD+ administration. On the other hand, when 10, 100, and 1,000 pmol of NMN was ICV injected once to the ND-fed C57BL/6 mice that were fasted overnight, a much lower level of food intake was observed 24 hours after the injection in the mice to which 10 pmol of NMN was injected, compared to the mice to which saline was injected (FIG. 2C).

Also, to examine an effect of the NAD+ administration through IP injection on the food intake, 0.3, 1, and 3 mg/kg of NAD+ was intraperitoneally administered once to the ND-fed C57BL/6 mice that were fasted overnight. As a result, it was confirmed that the food intake was significantly reduced at 4 hours and 24 hours after the NAD+ administration in the mice to which 1 mg/kg of NAD+ was injected, compared to the mice to which saline was injected (FIGS. 3A and 3B). On the other hand, when 30, 100, and 300 mg/kg of NMN was IP injected once to the C57BL/6 mice that were fasted overnight, a significantly reduced level of food intake was observed at 4 hours after the administration in the mice to which 300 mg/kg of NMN was injected, compared to the mice to which saline was injected, but no decrease in food intake was observed at 24 hours after the administration, compared to the control (FIGS. 3C and 3D).

In addition, an effect of the NAD+ administration on a change in body weight in the obese animal model was examined. A significantly reduced level of weight gain was observed at 24 hours after the NAD+ administration in the mice to which 0.2 pmol of NAD+ was injected, compared to the mice to which saline was ICV injected (FIG. 2B). On the other hand, there was no significant difference between the control group and the NMN-treated group in the change in body weight for 24 hours after the injection (FIG. 2D).

Additionally, an effect of chronic IP injection of NAD+ on the body weight was also examined (FIG. 4). The mice were divided into four groups (a group of ND-fed mice to which saline was IP injected, a group of ND-fed mice to which NAD+ was IP injected, a group of HFD-fed mice to which saline was IP injected, and a group of HFD-fed mice to which NAD+ was IP injected), and NAD+ was intraperitoneally administered to the mice at a dose of 0.3 mg/kg/day once a day for 4 weeks. The body weights of the ND-fed non-obese mice to which NAD+ was injected were not significantly different from those of the saline-administered counterpart during a NAD+ administration period, but a significant decrease in body weights of the HFD-fed obese mice was induced by the NAD+ administration.

Further, an effect of chronic IP injection of NAD+ on the feed intake was verified as follows. To examine a chronic effect of the NAD+ intraperitoneal injection on the feed intake, NAD+(0.3 mg/kg) or saline was administered to the following three groups of mice once a day for 4 weeks: an IP saline-injected group of mice fed the ND, an IP saline-injected group of mice fed the HFD, and an IP NAD+-injected group of mice fed the HFD.

In general, the ND-fed mice to which saline was injected exhibited an evident circadian rhythm of feed intake in which the mice were usually fed during the nighttime (corresponding to the daytime in humans) and hardly fed during the daytime. On the other hand, it was shown that the circadian rhythm of feed intake was disturbed as the feed intake during the daytime as well as the nighttime increased in the HFD-fed obese mice. The NAD+ injection to the obese mice did not significantly reduce the nighttime feed intake, but significantly reduced the daytime feed intake. Such research results showed that the NAD+ treatment was effective in reducing the disturbance of circadian rhythm of feed intake in individuals with obesity (FIG. 5B).

### Example 3

Effect of NAD+ Administration on Physical Activity in Obese Animal Model

To test an effect of habitual IP injection of NAD+(0.3 mg/kg/day) on mobility in an obese animal model, the mice were divided into three groups, that is, an IP saline-injected group of mice fed the ND, an IP saline-injected group of mice fed the HFD, and an IP NAD+-injected group of mice fed the HFD and the mobility thereof was evaluated. An evident circadian rhythm of physical activity was observed: the physical activity of the ND-fed normal control to which saline was injected was significantly higher during the nighttime (corresponding to the daytime in humans) than during the daytime. On the other hand, it was shown that the HFD-fed group of obese mice to which saline was administered had a disrupted circadian rhythm of physical activity, exhibiting significantly reduced nighttime physical activities compared to the control. The 4-week IP injection of NAD+ to the obese mice remarkably restored the nighttime mobility of the mice which had been reduced (see FIGS. 6A and 6B).

### Example 4

Effect of NAD+ Administration on Glucose Tolerance

FIG. 7 shows experimental results showing an effect of single IP injection of NAD+ on glucose tolerance. To examine an effect of single IP injection of NAD+ on the glucose tolerance, an oral glucose tolerance test was performed on ND-fed C57BL/6 mice that were fasted overnight. A single IP dose of NAD+(0.3, 1 and 3 mg/kg) was administered to the mice, and glucose (2 g/kg) was orally administered thereto after 30 minutes. Thereafter, blood glucose levels were measured at 15, 30, 60, and 120 minutes. The NAD+-injected mice had a much lower blood glucose level at 15 and 30 minutes after glucose loading, compared to the saline-injected mice.

While the present invention has been specifically shown and described with reference to preferred embodiments thereof, it will be understood by those skilled in the art to which the present invention belongs that various changes and modifications in form and details may be made therein without departing from the scope of the present invention. Therefore, the disclosed embodiments should be contemplated in descriptive senses only and not for purposes of limitation. Accordingly, the scope of the invention is defined not by the detailed description of the invention but by the appended claims, and all differences within the scope will be construed as being included in the present invention.

1. A method for preventing or treating obesity or impaired glucose tolerance, the method comprising:
administering nicotinamide adenine dinucleotide (NAD) or a pharmaceutically acceptable salt thereof to a target subject.

2. The method of claim 1, wherein the NAD or the pharmaceutically acceptable salt thereof controls a food intake pattern (including a food intake time and cycle) of an obese patient to reduce the intake of food.

3. The method of claim 1, wherein the NAD or the pharmaceutically acceptable salt thereof increases a physical activity of the obese patient.

4. The method of claim 1, wherein the NAD or the pharmaceutically acceptable salt thereof is administered in a form of intraperitoneal, intravascular or oral administration.

5. The method of claim 4, wherein the NAD or the pharmaceutically acceptable salt thereof is administered at a dose of 0.1 to 100 mg per unit weight (kg) of a target subject when the pharmaceutical composition is intraperitoneally or intravascularly administered.

6. The method of claim 1, wherein the administration is performed once to three times a day.

7. The method of claim 4, wherein the NAD or the pharmaceutically acceptable salt thereof is administered at a daily dose of 1 to 1,000 mg per unit weight (kg) of the target subject when the pharmaceutical composition is orally administered.

8. A method for preventing or improving symptoms of, obesity or impaired glucose tolerance, the method comprising:
administering a food composition containing NAD or a sitologically acceptable salt thereof.

9. The method of claim 8, wherein the NAD or the sitologically acceptable salt thereof controls a food intake pattern (including a food intake time and cycle) of an obese patient to reduce the intake of food.

10. (canceled)

11. (canceled)

12. (canceled)

13. (canceled)

14. (canceled)

15. (canceled)
